(19) United States
(12) Patent Application Publication
(10) Pub. No.: US 2026/0053411 A1
(43) Pub. Date: Feb. 26, 2026

(54) CALIBRATING GLOMERULAR FILTRATION RATE SENSING

(71) Applicant: Cardiac Pacemakers, Inc., St Paul, MN (US)

(72) Inventors: Pramodsingh Hirasingh Thakur, Woodbury, MN (US); Michael J. Kane, St Paul, MN (US); Ramesh Wariar, Blaine, MN (US); Yingbo Li, Woodbury, MN (US)

(21) Appl. No.: 19/304,588

(22) Filed: Aug. 19, 2025

Related U.S. Application Data
(60) Provisional application No. 63/685,255, filed on Aug. 20, 2024.

(51) Int. Cl. A61B 5/20; A61B 5/00; A61B 5/145; A61B 5/1455; A61B 5/1473; A61B 5/1495

(52) U.S. Cl. CPC A61B 5/201; A61B 5/1451; A61B 5/14546; A61B 5/1455; A61B 5/1473; A61B 5/1495; A61B 5/685; A61B 5/7271; A61B 2560/0223; A61B 2560/0462; A61B 2562/028

(57) ABSTRACT

Systems, devices, and methods involve approaches for determining a calibration value for an individual, estimating a creatinine level using a chemical sensor (e.g., based on an optical property of a chemical sensor), and estimating a glomerular filtration rate (GFR) based, at least in part, on the calibration value and the creatinine level.

### CROSS REFERENCE TO RELATED APPLICATION

This application claims priority to Provisional Application No. 63/685,255, filed Aug. 20, 2024, which is herein incorporated by reference in its entirety.

### TECHNICAL FIELD

Instances of the present disclosure relate to calibrating glomerular filtration rate sensing and using analyte sensing technology for evaluating performance of kidneys.

### BACKGROUND

A person's glomerular filtration rate can be used to measure aspects of a person's renal health.

### SUMMARY

In Example 1, a method includes determining a calibration value for an individual, estimating a creatinine level using a chemical sensor (e.g., based on an optical property of a chemical sensor), and estimating a glomerular filtration rate (GFR) based, at least in part, on the calibration value and the creatinine level.

In Example 2, the method of Example 1, wherein the chemical sensor is a wearable device that includes needles sized for access to interstitial fluid and a chemical indicator positioned within the needles.

In Example 3, the method of Example 2, wherein the optical property of the chemical indicator changes in response to different creatinine levels.

In Example 4, the method of any of Examples 1-3, further including: estimating the creatinine level based, at least in part, on the optical property contained in a digital image taken by an image sensor of a mobile computing device.

In Example 5, the method of any of Examples 1-4, wherein the determining the calibration value is based, at least in part, on the individual's response to a steady-state inulin test.

In Example 6, the method of any of Examples 1-4, wherein the determining the calibration value is based, at least in part, on the individual's response to a bolus inulin test.

In Example 7, the method of any of Examples 1-6, wherein the calibration value is a scaling factor applied to an output of a creatinine-to-GFR equation.

In Example 8, the method of any of Examples 1-7, wherein the estimating the GFR is further based, at least in part, on age and gender of the individual.

In Example 9, the method of any of Examples 1-8, wherein the estimating the GFR is further based, at least in part, on ethnicity of the individual.

In Example 10, the method of any of Examples 1-9, wherein the determining the calibration value comprises determining a slope.

In Example 11, the method of Example 10, wherein the slope minimizes an error between outputs of two calibration tests.

In Example 12, the method of Example 11, wherein the error is a root mean square (RMS) error.

In Example 13, a computer program product comprising instructions to cause one or more processors to carry out the steps of the method of claims 1-12.

In Example 14, a computer-readable medium having stored thereon the computer program product of Example 13.

In Example 15, a mobile device comprising the computer-readable medium of Example 14.

In Example 16, a system includes a mobile computing device with a processor, memory, and a user interface. The mobile computing device is programmed to: estimate a creatinine level using a chemical sensor (e.g., based on an optical property of a chemical sensor) and estimate GFR based, at least in part, on a calibration value and the creatinine level. The calibration value is based, at least in part, on multiple calibration tests.

In Example 17, the system of Example 16, further comprising: the chemical sensor, wherein the chemical sensor is a wearable device with a set of needles sized for access to interstitial fluid and a chemical indicator positioned within the set of needles, wherein the chemical indicator changes the optical property in response to different creatinine levels.

In Example 18, the system of Example 17, wherein the mobile computing device includes an image sensor.

In Example 19, the system of Example 18, wherein the mobile computing device is programmed to: determine the creatinine level based, at least in part, on the optical property of the chemical indicator.

In Example 20, the system of Example 16, wherein the mobile computing device includes an image sensor and is programmed to: determine the creatinine level based, at least in part, on the optical property contained in a digital image.

In Example 21, the system of Example 16, further including: the chemical sensor, wherein the chemical sensor is part of an implantable medical device and comprises a chemical indicator, wherein the chemical indicator changes optical properties in response to different creatine or potassium levels.

In Example 22, the system of Example 16, wherein the calibration value is a scaling factor or a coefficient applied to an output of a creatinine-to-GFR equation used for the estimate of the GFR.

In Example 23, the system of Example 16, wherein the estimate of the GFR is further based, at least in part, on age and gender of the individual.

In Example 24, the system of Example 23, wherein the estimate of the GFR is further based, at least in part, on ethnicity of the individual.

In Example 25, the system of Example 16, wherein the multiple calibration tests comprise a creatinine-based calibration test and a non-creatinine-based calibration test.

In Example 26, the system of Example 16, wherein the calibration value is based, at least in part, on a difference between outputs of the creatinine-based calibration test and a non-creatinine-based calibration test.

In Example 27, a method includes determining a calibration value for an individual, estimating a creatinine level using a chemical sensor (e.g., based on an optical property of a chemical sensor), and estimating GFR based, at least in part, on the calibration value and the creatinine level.

In Example 28, the method of Example 27, wherein the chemical sensor is a wearable device that includes needles sized for access to interstitial fluid and a chemical indicator positioned within the needles.

In Example 29, the method of Example 27, wherein the optical property of the chemical indicator changes in response to different creatinine levels.

In Example 30, the method of Example 27, further including: estimating the creatinine level based, at least in part, on the optical property contained in a digital image taken by an image sensor of a mobile computing device.

In Example 31, the method of Example 27, wherein the determining the calibration value is based, at least in part, on the individual's response to a steady-state inulin test.

In Example 32, the method of Example 27, wherein the determining the calibration value is based, at least in part, on the individual's response to a bolus inulin test.

In Example 33, the method of Example 27, wherein the calibration value is a scaling factor or a coefficient applied to an output of a creatinine-to-GFR equation.

In Example 34, the method of Example 27, wherein the estimating the GFR is further based, at least in part, on age and gender of the individual.

In Example 35, the method of Example 34, wherein the estimating the GFR is further based, at least in part, on ethnicity of the individual.

While multiple instances are disclosed, still other instances of the present disclosure will become apparent to those skilled in the art from the following detailed description, which shows and describes illustrative instances of the disclosure. Accordingly, the drawings and detailed description are to be regarded as illustrative in nature and not restrictive.

### BRIEF DESCRIPTION OF THE DRAWINGS

FIG. 1 is a schematic illustration of a chemical sensing system, in accordance with certain instances of the present disclosure.

FIG. 2 shows a block diagram of a method for use with one or more components of a chemical sensing system, in accordance with certain instances of the present disclosure.

FIG. 3 shows a graph that plots results of using different approaches for estimating glomerular filtration rates, in accordance with certain instances of the present disclosure.

FIG. 4 shows a schematic illustration of a computing device, in accordance with certain instances of the present disclosure.

FIGS. 5-7 show different views of various portions of a wearable chemical sensing device, in accordance with certain instances of the present disclosure.

FIGS. 8 and 9 show different views of various portions of an implantable medical device, in accordance with certain instances of the present disclosure.

FIG. 10 shows a block diagram with additional details of the computing device of FIG. 3, in accordance with certain instances of the present disclosure.

While the disclosed subject matter is amenable to various modifications and alternative forms, specific instances have been shown by way of example in the drawings and are described in detail below. The intention, however, is not to limit the disclosed subject matter to the particular instances described. On the contrary, the disclosed subject matter is intended to cover all modifications, equivalents, and alternatives falling within the scope of the disclosed subject matter as defined by the appended claims.

### DETAILED DESCRIPTION

Measuring a person's glomerular filtration rate (GFR) can be used to evaluate whether the person's kidneys are functioning properly. GFR measures the volume of fluid filtered from the renal glomerular capillaries into Bowman's capsule in kidneys. Current approaches for measuring GFR requires a person to visit a clinic or laboratory, be injected with a tracer, and potentially have their blood drawn.

Instead of measuring GFR using tracers, etc., creatinine can be measured and used to estimate a person's GFR. However, measuring creatinine may also require the person to visit a clinic and have their blood drawn. Further, each person's creatinine-to-GFR relationship may be unique because of various factors, so estimates of GFR based on creatinine measurements may be inaccurate.

Certain instances of the present disclosure are accordingly directed to approaches (e.g., systems, methods, devices) that use analyte sensing technology for evaluating analyte levels (e.g., creatinine levels) and calibration approaches that provide more accurate estimates of GFR using analyte levels on an individual-by-individual basis.

### Analyte Sensing System

Creatinine is an analyte that can be used as an indicator of a person's renal function. Creatinine is created as a byproduct of a muscle mass breakdown (e.g., muscle metabolism), and creatinine is supposed to be filtered (e.g., excreted) from the bloodstream by a person's kidneys. When renal function declines, less creatinine is excreted from the body, and serum concentrations of creatinine rise. Also, a person's renal function depends on adequate cardiac output, so a declining renal function indicates a decline in cardiac output. As such, measuring and monitoring creatinine levels can be used to evaluate whether a person's kidneys and heart are functioning properly.

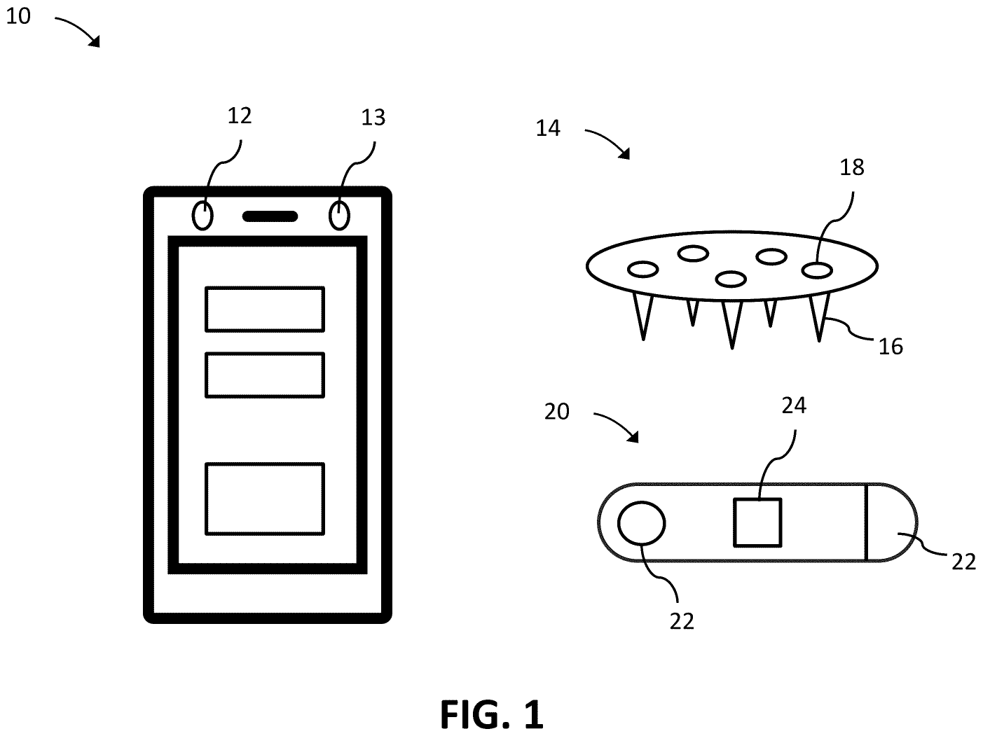

FIG. 1 shows a chemical sensing system 10 (hereinafter “the system 10” for brevity) with components that can be used to measure analytes such as creatinine. FIG. 1 shows the system 10 with schematic representations of two sets of components that can be used to measure analyte concentrations. Typically, measuring a patient's analyte concentrations requires drawing multiple blood samples from a patient at a clinic and processing the blood samples at a laboratory. Components of the chemical sensing system 10 can be used to measure analyte concentrations using a wearable device or an implantable medical device. Although the chemical sensors shown in FIG. 1 and described below are optical-based chemical sensors, other types of chemical sensor approaches can be used (e.g., electrical-based chemical sensors such as those that use electrodes and measure potential differences, and the like).

For the wearable approach, the system 10 can include a device (e.g., a mobile computing device described further herein) with an image sensor 12 and a chemical sensing device 14. The image sensor 12 (e.g., a charge coupled device, a complementary metal oxide semiconductor, or other devices that can capture an image) can be part of a camera, smart phone, or other device able to capture an image (e.g., a digital image). In certain instances, the image sensor 12 and the chemical sensing device 14 are integrated into a single device, and in other instances the image sensor 12 and the chemical sensing device 14 are separate devices. In instances where the image sensor 12 is part of a mobile computing device such as a smart phone, the smart phone can store, operate, or otherwise access a program (e.g., a phone application) that processes an image (of the chemical sensing device 14) taken by the image sensor 12 and determines estimates of one or more analyte concentrations of the patient. In other instances, the image sensor 12 is part of a dedicated readout device or part of a camera. The system 10 can include one or more light sources 13, which can be part of the same device as the image sensor 12 or which can be part of a separate component. The one or more light sources 13 can generate light (e.g., emit visible light, ultraviolet light, monochromatic light (red, green, blue)).

The chemical sensing device 14 can be a wearable device (e.g., an exterior device and not an implantable device) such as a device that includes (or is part of) a strap (e.g., an armband strap), a patch (e.g., a torso patch), or another type of device that can be coupled to a patient's skin. For simplicity, the chemical sensing device 14 is hereinafter referred to as the “patch 14” although other types of wearable devices can use the chemical sensing technology described herein.

In certain instances, the patch 14 is a transdermal patch that includes a mechanism (e.g., needles 160 for accessing a patient's interstitial fluid. For example, multiple needles 16 (e.g., microneedles) can be sized to access a patient's interstitial fluid. The patch 14 can also include multiple chemical indicators 18, each of which changes optical properties (e.g., fluorimetric properties, colorimetric properties) with changes in concentration of a certain analyte in the interstitial fluid. As described in more detail herein, the image sensor 12 can be used to capture an image (e.g., a digital image) of the chemical indicators 18, and the image can be processed and analyzed to determine respective concentrations of targeted analytes. In certain instances, the patch 14 includes one type of chemical indicator 18 (e.g., to help determine concentration of one type of analyte), but in other instances the patch 14 includes multiple types of chemical indicators.

For the implantable approach, the system 10 can include an implantable medical device 20, which includes one or more electrodes 22 and a chemical sensor assembly 24. The electrodes 22 can comprise a conductive material and be configured to sense cardiac activation signals. The chemical sensor assembly 24 can include a sensing element with a polymeric matrix permeable to analytes such as creatinine and/or potassium. The sensing element can include an interior volume with various chemical indicators (e.g., beads for detecting an ion concentration of a bodily fluid when implanted in the body disposed within an interior volume). Analytes can diffuse through an outer barrier layer and onto and/or into the chemical indicators where the analytes can bind with ion selective sensors to produce an optical response (e.g., a change in optical properties such as a change in concentration, a fluorimetric response, a colorimetric response). The optical response can be monitored and used to estimate analyte levels. The estimated analyte levels can be used by a computing device to monitor and evaluate a person's kidney and/or cardiac performance.

### Methods

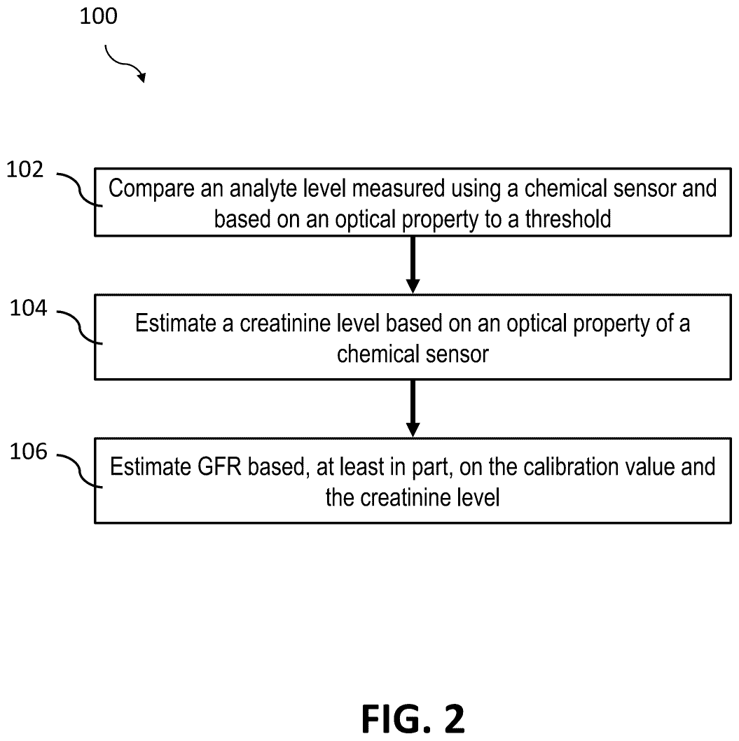

FIG. 2 outlines a method that can be used in connection with one or more components of the system 10 of FIG. 1 or other components described herein.

FIG. 2 shows a block diagram of a method 100 for estimating GFR based, at least in part, on creatinine levels (e.g., serum creatinine concentrations) and a calibration value. In certain instances, the method 100 is carried out by one or more computing devices such as a mobile phone, a tablet, a laptop computer, a desktop computer, a server. For example, the method 100 could be carried out by an application that is downloadable to the device, operated on the device, and accessible by a patient, their physician, their clinic, and the like. Different steps of the method 100 can be carried out by a combination of devices. For example, certain steps involving determining a calibration value may involve a first set of one or devices, while the remaining steps (e.g., estimating GFR) may involve a second set of one or more devices.

The method 100 includes determining a calibration value for an individual (block 102 in FIG. 2). As will be described in more detail below, the calibration value is ultimately used for more accurately estimating GFR using creatinine levels. Each person's creatinine-to-GFR relationship may be unique because of various factors (e.g., diet, muscle mass, age, gender, ethnicity). A calibration value can be used to account for the various factors that affect an individual's creatinine-to-GFR relationship.

Determining a calibration value can involve multiple steps. In certain instances, before the calibration value is determined, a person is subjected to one or more calibration tests. The calibration tests are used to determine the person's GFR using different approaches. The different approaches can include a non-creatinine-based calibration test and a creatinine-based calibration test. The output (e.g., estimated GFR) of the non-creatinine-based calibration test can be compared to the output (e.g., estimated GFR) of the creatinine-based calibration test. The calibration value can be calculated such that—when the calibration value is applied to (e.g., multiplied by) the output of the creatinine-based calibration test—the calibrated output is consistent with or closely aligns with the output of the non-creatinine-based calibration test. Put another way, the calibration value can be used so that creatinine levels can be used to more accurately estimate GFR without needing to use non-creatinine measurement methods every time GFR is estimated. Non-creatinine measurement methods require more time and resources.

In certain instances, the non-creatinine-based calibration test involves injecting the person with a tracer (e.g., inulin, radioactive material) and monitoring how their kidneys function. In some examples, injecting the person with inulin involves injecting the person with a bolus of inulin. This may be referred to as a bolus inulin test, which involves collecting multiple blood samples (e.g., 10-12 samples) after the injection and plotting plasma concentration level over time to determine a plasma disappearance curve. The plasma clearance of inulin can be calculated by dividing the dose by the area under the plasma disappearance curve. In other examples, injecting the person with inulin involves a steady-state or continuous injection. This may be referred to as a steady-state test, which does not involve blood draws. Using this approach, once the inulin has reached a steady state in the plasma and the volume of distribution is saturated, the rate of elimination of the inulin will equal the rate of infusion (RI). The clearance of the marker will equal RI divided by Px (the plasma concentration of inulin).

In certain instances, the non-creatinine-based calibration test involves using a chemical sensor to measure one or more creatinine levels. In some instances, the one or more creatinine levels are used as an input into an equation that estimates GFR based, at least in part, on a creatinine level. One equation that can be used is:

$$GFR = 141 \times {min ( {S}_{cr} / κ , 1 )}^{a} \times {max ( {S}_{cr} / κ , 1 )}^{- 1.209} \times {0.993}^{Age} \times ( 1.018 if female ) \times ( 1.159 if African American )$$

Where S_{cr} is serum creatinine in mg/dl, κ is 0.7 for females and 0.9 for males, α is −0.329 for females and −0.411 for males, min indicates the minimum of S_{cr}/κ or 1, max indicates the maximum of S_{cr}/κ or 1, and Age is the person's age in years. Another equation that can be used is:

$$GFR = 175 \times {( {S}_{cr} )}^{- 1.154} \times {( Age )}^{- 0.203} \times ( 0.742 if female ) \times ( 1.212 if African American )$$

Other equations can be used to estimate GFR based, at least in part, on a creatinine level.

To determine the calibration value (per block 102 in FIG. 2), a coefficient or scaling factor can be calculated such that the GFR estimated by a creatinine-to-GFR equation is consistent with the GFR estimated from the non-creatinine-based calibration test. For example, the calibration value can be a coefficient or scaling factor that is multiplied by the output of the creatinine-to-GFR equation such that using the creatinine-to-GFR equation results in an estimated GFR that aligns with the GFR estimated by the non-creatinine-based approach. The determined calibration value can then be used with (e.g., multiplied by) the output of the creatinine-to-GFR equation to estimate GFR using a chemical sensor and measured creatinine levels.

As such, in certain instances, the calibration value is a scaling factor or a coefficient used in connection with a creatinine-to-GFR equation. The calibration value can be a scaling factor or coefficient that reduces (e.g., minimizes) error (e.g., differences) between the outputs of the non-creatinine-based calibration test and the creatinine-based calibration test. In certain instances, the error being minimized or otherwise reduced is a root mean square (RMS) error.

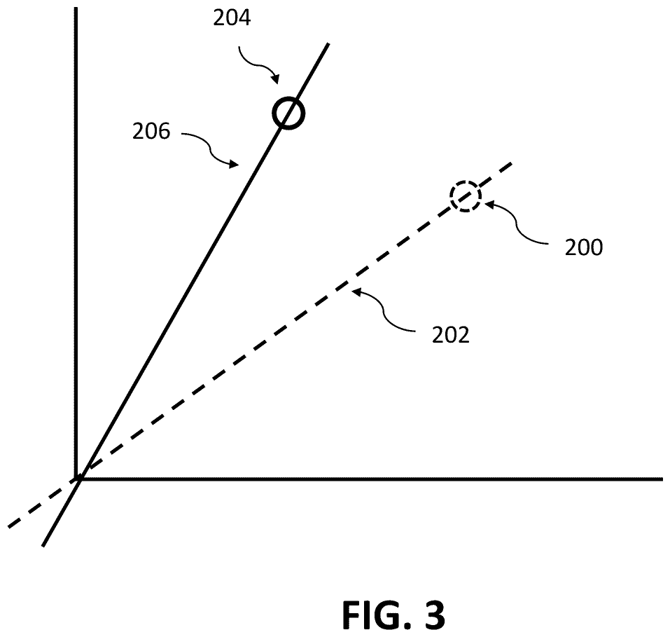

FIG. 3 visually shows the effect of using a calibration value. In FIG. 3, two estimated GFR values are shown and represented by circles.

One estimated GFR value was calculated using a creatinine-to-GFR equation and is represented by a circle associated with reference number 200. A dashed line 202 extends along a first slope between the origin (0,0) and the circle 200.

The other estimated GFR value was determined using a non-creatinine-based calibration test and is represented by a circle associated with reference number 204. A solid line 206 extends along a second slope between the origin (0,0) and the circle 204.

As can be seen in FIG. 3, the two estimates are different and inconsistent with each other. The slope of the dashed line 202 is less than the slope of the solid line 206, which indicates that the creatinine-to-GFR equation is underestimating the individual's GFR.

To align the GFR estimation 200 from the creatinine-to-GFR equation with the GFR estimation 204 from the non-creatinine-based calibration test, a calibration value (e.g., a coefficient or scaling value) can be applied to the creatinine-to-GFR equation. The calibration value can be applied such that the slope of the dashed line 202 matches (or at least substantially matches) the slope of the solid line 206. To increase the slope, the calibration value can be >1; and to decrease the slope, the calibration value can be <1. Although only one output of each GFR approach is shown in FIG. 3, multiple outputs from one or both approaches can be compared and used to determine a calibration value. Using and reconciling multiple outputs can increase the accuracy of the calibration value.

In certain instances, a new calibration value is calculated periodically. Because a person's creatinine-to-GFR relationship can change as various factors change (e.g., diet, muscle mass), a new calibration value may need to be calculated to increase the accuracy of creatinine-to-GFR equations.

Referring back to the method 100 of FIG. 2, the method 100 includes estimating a creatinine level using a chemical sensor (e.g., based on an optical property of a chemical sensor) (block 104 in FIG. 2). The chemical sensor can be the types of chemical sensors described in connection with FIG. 1 and herein. A chemical indicator that is part of the chemical sensor can be in communication with a person's blood such that one or more optical properties of the chemical indicator can be monitored to determine a level (e.g., concentration) of creatinine.

In certain instances, estimating a creatinine level (and therefore GFR) occurs periodically (e.g., every 30 minutes, once an hour, once a day) or on demand (e.g., when a patient or physician initiates the comparison). Although the chemical sensors may react in real-time (e.g., the chemical indicators change optical properties in real-time as analyte levels change in real-time), transmission of or calculating a creatinine level can occur less often to save computing and battery resources and because creatinine levels may not change drastically minute-by-minute.

The method 100 further includes estimating GFR based, at least in part, on the calibration value and the creatinine level (block 106 in FIG. 2). A person's kidneys are supposed to maintain a constant renal blood flood via autonomic and autoregulation mechanisms. Once a person's sustained GFR is below 60 mL/min/1.73 m², it can be an indicator that the person has chronic kidney disease.

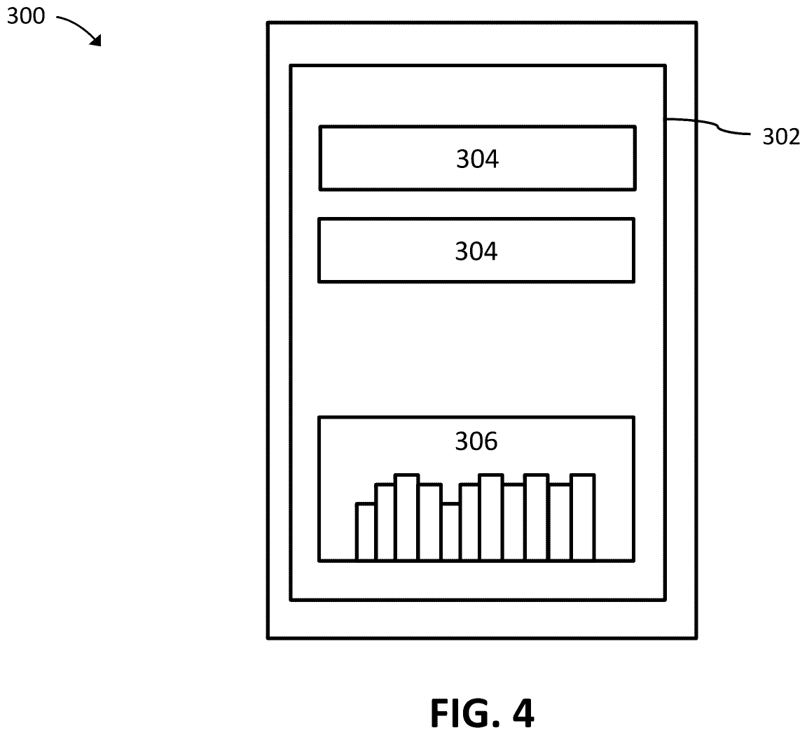

In certain instances, the estimated GFR is compared to a threshold. If the estimated GFR reaches the threshold, an alert can be displayed on a user interface of a computing device (e.g., a mobile computing device). FIG. 4 shows an example computing device 300 such as a mobile phone or tablet that includes a user interface 302. The device 300 can operate an application such that the user interface 302 displays various screens, icons, and/or buttons to help the user navigate and use the application. One or more graphics, pictures, videos, and/or text passages 304 can be used to display the alert. Any of the data (e.g., estimated creatinine, estimated GFR) described herein—whether historical or in real-time—can be displayed on the user interface 302 of the computing device 300. For example, the user interface 302 can be arranged to include a window 306 in which one or more sets of data can be displayed (e.g., levels as a function of time).

### Wearable Chemical Sensor

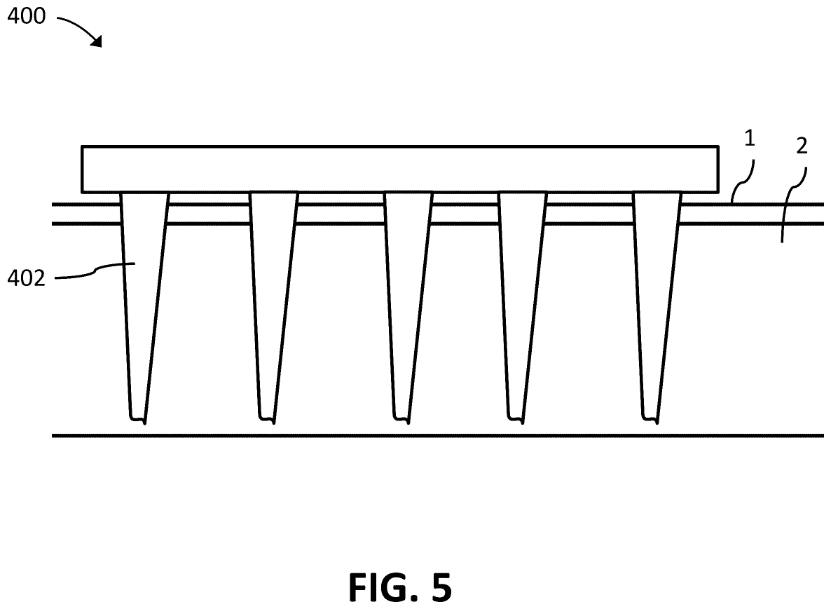

FIG. 5 shows a schematic side view of a wearable chemical sensing device 400. For simplicity, the device 400 is hereinafter referred to as the “patch 400” although other types of wearable devices can use the chemical sensing technology described herein. The patch 400 can be coupled to the patient's skin 1 such that needles 402 pierce through the outer layer of skin 1 and extend into the patient's interstitial fluid space 2. The needles 402 can have openings that are exposed to the patient's interstitial fluid (and therefore analytes within the patient's interstitial fluid).

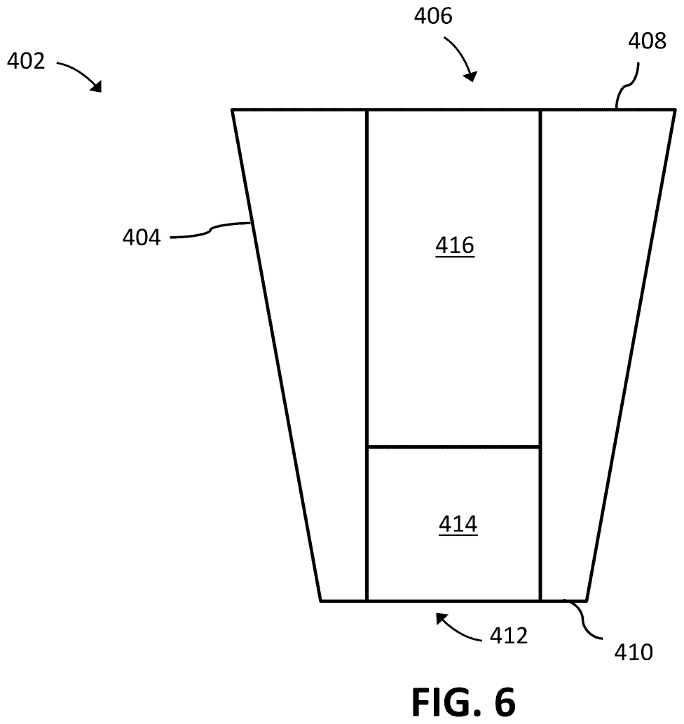

FIG. 6 shows a schematic side view of one of the needles 402 of the patch 400. In certain instances, the needles 402 are hollow needles such that each needle 402 includes an outer needle structure 404 that surrounds an opening 406 (e.g., a central thru-hole within the needle 402). The opening 406 can extend from a proximal end 408 of the needle 402 to a distal end 410 of the needle 402. An aperture 412 is located at or near the distal end 410 of the needle 402 such that the opening 406 is exposed to interstitial fluid.

Also at or near the distal end 410 of the needle 402 is a membrane 414 (e.g., a diffusion membrane) that is positioned within the needle 402. The membrane 414 protects tissue from direct interaction or exposure to a chemical indicator 416 that is also positioned within the needle 402. The membrane 414 can be formed from a permeable material, such as an ion permeable polymeric matrix material. In some instances, the membrane 414 can be permeable to sodium ions, potassium ions, hydronium ions, creatinine, urea, and various additional analytes. As referenced above, the cover membrane of the sensing element can be formed of a permeable material. In some embodiments, the cover membrane can be formed from an ion-permeable polymeric matrix material. Suitable polymers for use as the ion-permeable polymeric matrix material can include, but are not limited to, polymers forming a hydrogel. Hydrogels herein can include homopolymeric hydrogels, copolymeric hydrogels, and multipolymer interpenetrating polymeric hydrogels. Hydrogels herein can specifically include nonionic hydrogels. In certain instances, the membrane 414 includes an active agent disposed therein including, but not limited to anti-inflammatory agents, angiogenic agents, and the like.

The particular type (e.g., type of ion selectivity) and length of membrane can vary by needle 402. For example, one set of needles 402 can include a membrane 414 that is permeable to creatinine ions, while another set of needles 402 includes a membrane 414 that is permeable to potassium or sodium ions, and so on. In other examples, the membrane 414 is agnostic to a particular type of ion. The membrane 414 is positioned such that analytes must pass through the membrane 414 before reaching the chemical indicator 416. The membrane 414 material used will affect how fast an analyte travels between interstitial fluid and the chemical indicator 416.

The chemical indicator 416 comprises a material that changes properties (e.g., optical properties such as absorption, transmission, scattering, fluorescence) with changes in concentration of a given analyte. As one example, the chemical indicator 416 can comprise a creatinine select compound that changes optical properties in response to the creatinine select compound binding to creatine. As another example, the chemical indicator 416 can comprise a creatinine deiminase enzyme covalently bound to a substrate and a pH-indicating compound in ionic communication with the creatinine deiminase enzyme. In this example, the chemical indicator 416 can change optical properties in response to changes in creatinine concentrations in vivo. As another example, the chemical indicator 416 can comprise a creatinine select compound, and a pH-indicating compound in ionic communication with bodily fluid. In this example, the chemical indicator 416 can change optical properties in response to changes in creatinine concentrations in vivo. And in this example, the chemical indicator 416 may also comprise a mechanism to change local pH within the chemical indicator 416.

In certain instances, color of the chemical indicator 416 comprises the sum of the absorption, transmission, reflectance, and fluorescence properties of the chemical indicator material. Put another way, the chemical indicator 416 can comprise a material that changes optical properties with changes in concentration of a given analyte—and such optical properties can be measured by analyzing an image of the chemical indicator 416. In certain instances, the chemical indicator 416 has a minimum thickness or height along a longitudinal axis of a needle of 0.15-0.60 mm (e.g., 0.50-0.60 mm). In certain instances, the chemical indicator 416 comprises a slurry or a film.

In certain instances, the chemical indicator 416 is formed of a lipophilic indicator dye (e.g., a lipophilic fluorescent indicator dye or a lipophilic colorimetric indicator dye). Lipophilic indicator dyes can include, but are not limited to, ion selective sensors such as ionophores or fluorophores. In certain instances, ionophores can include sodium-specific ionophores, potassium-specific ionophores, calcium-specific ionophores, magnesium-specific ionophores, and lithium-specific ionophores. In certain instances, fluorophores can include lithium-specific fluorophores, sodium-specific fluorophores, and potassium-specific fluorophores.

Compositions of the chemical indicator 416 can include components (or response elements) that are configured for a colorimetric response, a photoluminescent response, or another optical sensing modality. For example, the chemical indicator 416 can include an element that changes color based on binding with or otherwise complexing with a specific chemical analyte. As one specific example, creatinine reacts with a molecule which changes pH and color on the indicator. In some instances, the chemical indicator 416 can include a complexing moiety and a colorimetric moiety. Those moieties can be a part of a single chemical compound (e.g., a non-carrier-based system) or can be separated on two or more different chemical compounds (e.g., a carrier-based system). The colorimetric moiety can exhibit differential light absorbance on binding of the complexing moiety to an analyte.

Some of the chemical indicators 416 may not require a separate compound to both complex an analyte of interest and produce an optical response. By way of example, in some instances, the response element can include a non-carrier optical moiety or material wherein selective complexation with the analyte of interest directly produces either a colorimetric or fluorescent response. As an example, a fluoroionophore can be used and is a compound including both a fluorescent moiety and an ion complexing moiety. As merely one example, (6,7-[2.2.2]-cryptando-3-[2″-(5″-carboethoxy)thiophenyl]coumarin, a potassium ion selective fluoroionophore, can be used (and in some cases covalently attached to polymeric matrix or membrane) to produce a fluorescence-based K+ non-carrier response element. An exemplary class of fluoroionophores are the coumarocryptands. Coumarocryptands can include lithium specific fluoroionophores, sodium specific fluoroionophores, and potassium specific fluoroionophores. For example, lithium specific fluoroionophores can include (6,7-[2.1.1]-cryptando-3-[2″-(5″-carboethoxy) furyl]coumarin. Sodium specific fluoroionophores can include (6,7-[2.2.1]-cryptando-3-[2″-(5″-carboethoxy) furyl]coumarin. Potassium specific fluoroionophores can include (6,7-[2.2.2]-cryptando-3-[2″-(5″-carboethoxy) furyl]coumarin and (6,7-[2.2.2]-cryptando-3-[2″-(5″-carboethoxy)thiophenyl]coumarin.

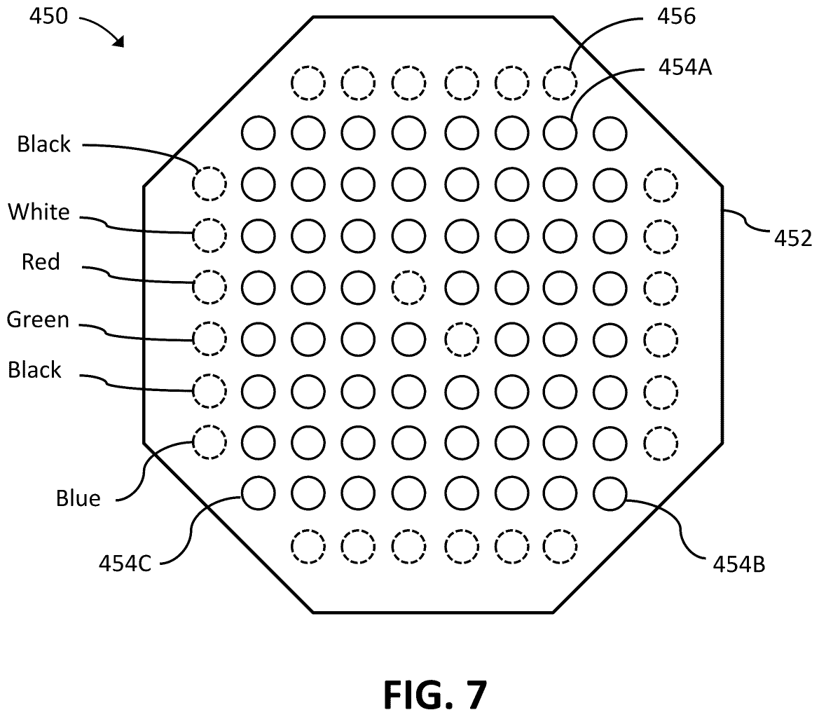

FIG. 7 shows a top view of a patch 450. The arrangement shown in FIG. 7 can be used in connections with the needles, membranes, chemical indicators, and layers, etc., described above with respect to the patch 400. The view shown in FIG. 7 is the type of view of a patch that an image sensor would capture in a digital image while the patch is coupled to the patient. The digital image can capture the colors of the chemical indicators (and color references) such that the colors can be analyzed to determine concentrations of one or more analytes of the patient.

FIG. 7 shows a patch 450, which includes a window 452 through which various components of the patch 100 can be viewed. In particular, the window 452 allows chemical indicators 454A-C of the patch 450 to be viewed. The chemical indicators 454A-C can be positioned in needles (e.g., hollow needles) such as the needles described above. As such, each chemical indicator 454A-C can be associated with its own needle.

A first set of needles can include a first type of chemical indicator 454A such as a chemical indicator that changes in color with changes in concentration of a first analyte (e.g., creatinine). A second set of needles can include a second type of chemical indicator sodium 454B such as a chemical indicator that changes in color with changes in concentration of a second analyte (e.g., potassium). A third set of needles can include a third type of chemical indicator 454C such as a chemical indicator that changes in color with changes in concentration of a third analyte (e.g., sodium). The respective colors of the chemical indicators can be used to estimate the respective concentrations of analytes in a patient's interstitial fluid.

In certain instances, each of the first type of chemical indicators 454A are positioned near or next to each other, each of the second type of chemical indicators 454B are positioned near or next to each other, and so on. The overall number of chemical indicators (and therefore the number of needles) and the number of different sets of types of chemical indicators on a given patch can be fewer or greater than that shown in FIG. 7. For example, the patch 450 could include a single type of chemical indicator selected for a single type of analyte. The relative positions of the chemical indicators 454A-C can vary from that shown in FIG. 7, and the specific shape of the chemical indicators 454A-C (as seen from a top view) can vary from the circular shapes shown in FIG. 7.

The patch 450 can also include color references 456. The color references 456 are shown in dotted lines in FIG. 7. The color references 456 can help with calibrating, correcting, and/or processing the digital image of the chemical indicators 454A-C such that an accurate estimate of the color of the chemical indicators 454A-C can be determined. For example, because the color of the color references 456 is known, the color of the chemical indicators 454A-C can be more accurately estimated as the patch 450 is positioned in different lighting (e.g., in direct sunlight, in a shadow, partially shaded, and the like). As such, the color references 456 can act as a color index or reference point for correcting for changes in color caused by ambient light.

In certain instances, some of the color references 456 are black, others white, others red, others green, others blue. Although most of the color references 456 in FIG. 7 are shown around a perimeter of the patch (e.g., with the chemical indicators 454A-C positioned within the perimeter), other positions and arrangements of the color references 456 can be utilized in the patch 450. The overall number and the specific shape of the color references 456 (as seen from a top view) can vary from the circular or dot shape shown in FIG. 7.

Using the patches described herein, analyte concentrations can be estimated. For example, a digital image of a patch attached to a patient can be taken by a camera and an analyte concentration can be estimated based on a color of one or more chemical indicators. In certain instances, estimating the analyte concentrations involves calculating an analyte concentration for multiple chemical indicators and then applying a mathematical operation (e.g., averaging, voting) to determine the respective analyte concentrations. The analyte concentration estimations can be further based on corrections that are determined using color reference sections of the patch. Each set or grouping of chemical indicators from the digital image can be processed and their respective colors compared to a table, library, mapping, index, etc. that associates a given color of chemical indicator to a given concentration level. In certain instances, the process of estimating analyte concentrations is carried out by an application stored on and operated by a smart phone. In other instances, some or all steps can be carried out by a server or other computing system besides a smart phone that can access digital images of a patch and be programmed to determine estimated analyte concentration levels based on colors of chemical indicators shown in the digital image.

U.S. patent application Ser. No. 18/774,681 describes additional details of a wearable chemical sensing system and is herein incorporated by reference in its entirety.

### Implantable Chemical Sensor

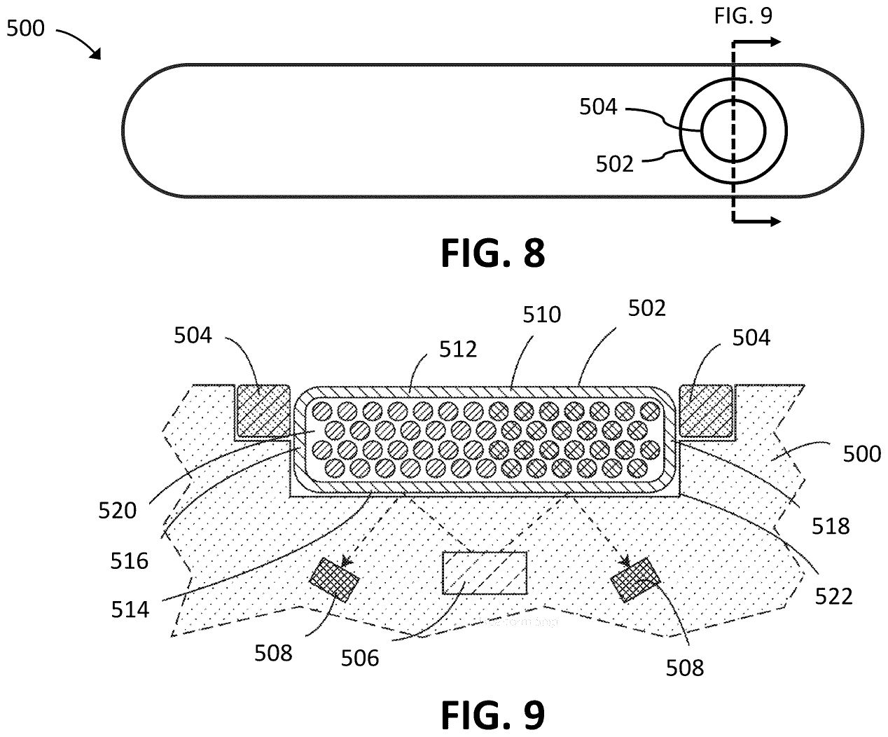

FIGS. 8 and 9 show an implantable medical device (IMD) 500 with a chemical sensor assembly.

FIG. 8 shows a top-down view of the IMD 500 with a sensing element 502 and an active agent eluting material 504 (or active agent eluting matrix) disposed around the outer perimeter of sensing element 502. In some instances, the active agent eluting material 504 forms a ring structure around the outer perimeter of sensing element 502. It will be appreciated that the sensing element 502 and the active agent eluting material 504 can have different geometric shapes and sizes.

FIG. 9 shows a cross-sectional view of a portion of the IMD 500 and its chemical sensor assembly. The IMD 500 includes an optical excitation assembly 506 and optical detection assembly 508. The sensing element 502 can include an outer barrier layer 510 formed, in full or in part, from a permeable material, such as an ion permeable polymeric matrix material. The outer barrier layer 510 can form a top 512, a bottom 514, and opposed sides 516 and 518 to surround an interior volume 520 of the sensing element 502. In certain instances, at least the top 512 of outer barrier layer 510 is permeable to sodium ions, potassium ions, hydronium ions, creatinine, urea, and the like. The outer barrier layer 510 can also include an active agent disposed therein including, but not limited to anti-inflammatory agents, angiogenic agents, and the like. The housing of the IMD 500 can include a recessed pan 522 into which the sensing element 502 fits. In some embodiments, implantable housing can define an aperture occluded by a transparent member. The transparent member can be a glass (including but not limited to borosilicate glasses), a polymer or other transparent material. The aperture can be disposed at the bottom of the recessed pan. The aperture can provide an interface allowing for optical communication between sensing element and the optical excitation and optical detection assemblies. It will be appreciated that outer barrier layer, or portions thereof, can be made from a transparent polymer matrix material to allow for optical communication between the sensing element 502 and optical excitation 506 and optical detection 508 assemblies.

The optical excitation assembly 506 can be designed to illuminate the sensing element 502. The optical excitation assembly 508 can include a light source such as a light emitting diode (LED), vertical-cavity surface-emitting lasers (VCSELs), electroluminescent (EL) devices, and the like. The optical detection assembly 508 can include a component selected from the group consisting of a photodiode, a phototransistor, a charge-coupled device (CCD), a junction field effect transistor (JFET) optical sensor, a complementary metal-oxide semiconductor (CMOS) optical sensor, an integrated photo detector integrated circuit, a light to voltage converter, and the like.

Various indicator beads can be positioned in the interior volume 520. The indicator beads can be used for detecting an ion concentration of a bodily fluid. For example, the indicator beads can include a polymeric support material and one or more ion selective sensing components as described more fully below. Analytes such as creatinine, potassium ion, sodium ion, hydronium ion, and the like, can diffuse through the top of the outer barrier layer and onto and/or into the indicator beads where they can bind with the ion selective sensors to produce a change in optical properties (e.g., a fluorimetric response, a colorimetric response).

U.S. Patent App. Pub. No. 2018/0344218 describes additional details of an implantable medical device with a chemical sensor assembly and is herein incorporated by reference in its entirety.

### Computing Device

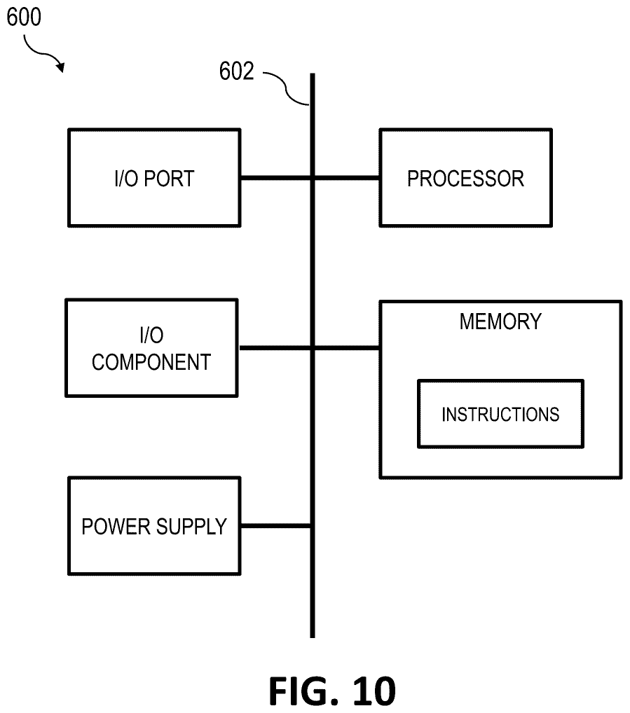

FIG. 10 is a block diagram depicting additional details of the computing device 300 shown in FIG. 4. The computing device 300 may include any type of computing device suitable for implementing aspects of instances of the disclosed subject matter. Examples of computing devices include specialized computing devices or general-purpose computing devices such as workstations, servers, laptops, desktops, tablet computers, hand-held devices, smartphones, general-purpose graphics processing units (GPGPUs), and the like.

In instances, the computing device 300 includes a bus 302 that, directly and/or indirectly, couples one or more of the following devices: a processor, a memory, an input/output (I/O) port, an I/O component, and a power supply. Any number of additional components, different components, and/or combinations of components may also be included in the computing device 300.

The bus 302 represents what may be one or more busses (such as, for example, an address bus, data bus, or combination thereof). Similarly, in instances, the computing device 300 may include a number of processors, a number of memory components, a number of I/O ports, a number of I/O components, and/or a number of power supplies. Additionally, any number of these components, or combinations thereof, may be distributed and/or duplicated across a number of computing devices.

In instances, the memory includes computer-readable media in the form of volatile and/or nonvolatile memory and may be removable, nonremovable, or a combination thereof. Media examples include random access memory (RAM); read only memory (ROM); electronically erasable programmable read only memory (EEPROM); flash memory; optical or holographic media; magnetic cassettes, magnetic tape, magnetic disk storage or other magnetic storage devices; data transmissions; and/or any other medium that can be used to store information and can be accessed by a computing device. In instances, the memory stores computer-executable instructions for causing the processor to implement aspects of instances of components discussed herein and/or to perform aspects of instances of methods and procedures discussed herein. The memory can comprise a non-transitory computer readable medium storing the computer-executable instructions.

The computer-executable instructions may include, for example, computer code, machine-useable instructions, and the like such as, for example, program components capable of being executed by one or more processors (e.g., microprocessors) associated with the computing device 300. Program components may be programmed using any number of different programming environments, including various languages, development kits, frameworks, and/or the like. Some or all of the functionality contemplated herein may also, or alternatively, be implemented in hardware and/or firmware.

According to instances, for example, the instructions may be configured to be executed by the processor and, upon execution, to cause the processor to perform certain processes. In certain instances, the processor, memory, and instructions are part of a controller such as an application specific integrated circuit (ASIC), field-programmable gate array (FPGA), and/or the like. Such devices can be used to carry out the functions and steps described herein.

The I/O component may include a presentation component configured to present information to a user such as, for example, a display device, a speaker, a printing device, and/or the like, and/or an input component such as, for example, a microphone, a joystick, a satellite dish, a scanner, a wireless device, a keyboard, a pen, a voice input device, a touch input device, a touch-screen device, an interactive display device, a mouse, and/or the like.

The devices and systems described herein can be communicatively coupled via a network, which may include a local area network (LAN), a wide area network (WAN), a cellular data network, via the internet using an internet service provider, and the like.

Aspects of the present disclosure are described with reference to flowchart illustrations and/or block diagrams of methods, devices, systems and computer program products. It will be understood that each block of the flowchart illustrations and/or block diagrams, and combinations of blocks in the flowchart illustrations and/or block diagrams, can be implemented by computer program instructions.

Various modifications and additions can be made to the exemplary embodiments discussed without departing from the scope of the present invention. For example, while the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

We claim:

1. A system comprising:
a mobile computing device including a processor, memory, and a user interface, wherein the mobile computing device is programmed to:
estimate a creatinine level based on an optical property of a chemical sensor; and
estimate a glomerular filtration rate (GFR) based, at least in part, on a calibration value and the creatinine level, wherein the calibration value is based, at least in part, on multiple calibration tests.

2. The system of claim 1, further comprising: the chemical sensor, wherein the chemical sensor is a wearable device with a set of needles sized for access to interstitial fluid and a chemical indicator positioned within the set of needles, wherein the chemical indicator changes the optical property in response to different creatinine levels.

3. The system of claim 2, wherein the mobile computing device includes an image sensor.

4. The system of claim 3, wherein the mobile computing device is programmed to: determine the creatinine level based, at least in part, on the optical property of the chemical indicator.

5. The system of claim 1, wherein the mobile computing device includes an image sensor and is programmed to: determine the creatinine level based, at least in part, on the optical property contained in a digital image.

6. The system of claim 1, further comprising: the chemical sensor, wherein the chemical sensor is part of an implantable medical device and comprises a chemical indicator, wherein the chemical indicator changes optical properties in response to different creatine or potassium levels.

7. The system of claim 1, wherein the calibration value is a scaling factor or a coefficient applied to an output of a creatinine-to-GFR equation used for the estimate of the GFR.

8. The system of claim 1, wherein the estimate of the GFR is further based, at least in part, on age and gender of the individual.

9. The system of claim 8, wherein the estimate of the GFR is further based, at least in part, on ethnicity of the individual.

10. The system of claim 1, wherein the multiple calibration tests comprise a creatinine-based calibration test and a non-creatinine-based calibration test.

11. The system of claim 1, wherein the calibration value is based, at least in part, on a difference between outputs of the creatinine-based calibration test and a non-creatinine-based calibration test.

12. A method comprising:
determining a calibration value for an individual;
estimating a creatinine level based on an optical property of a chemical sensor; and
estimating a glomerular filtration rate (GFR) based, at least in part, on the calibration value and the creatinine level.

13. The method of claim 12, wherein the chemical sensor is a wearable device that includes needles sized for access to interstitial fluid and a chemical indicator positioned within the needles.

14. The method of claim 12, wherein the optical property of the chemical indicator changes in response to different creatinine levels.

15. The method of claim 12, further comprising: estimating the creatinine level based, at least in part, on the optical property contained in a digital image taken by an image sensor of a mobile computing device.

16. The method of claim 12, wherein the determining the calibration value is based, at least in part, on the individual's response to a steady-state inulin test.

17. The method of claim 12, wherein the determining the calibration value is based, at least in part, on the individual's response to a bolus inulin test.

18. The method of claim 12, wherein the calibration value is a scaling factor or a coefficient applied to an output of a creatinine-to-GFR equation.

19. The method of claim 12, wherein the estimating the GFR is further based, at least in part, on age and gender of the individual.

20. The method of claim 19, wherein the estimating the GFR is further based, at least in part, on ethnicity of the individual.
